(19) United States
(12) Patent Application Publication
(10) Pub. No.: US 2025/0359935 A1
(43) Pub. Date: Nov. 27, 2025

(54) PREDICTION OF BONE BASED ON POINT CLOUD

(71) Applicant: Howmedica Osteonics Corp., Mahwah, NJ (US)

(72) Inventors: Yannick Morvan, Saint Renan (FR); Jérôme Ogor, Brest (FR); Jean Chaoui, Lyon (FR); Julien Ogor, Brest (FR); Thibaut Nico, Brest (FR)

(21) Appl. No.: 18/872,550

(22) PCT Filed: Jun. 2, 2023
(86) PCT No.: PCT/US2023/024330

§ 371 Date: Dec. 6, 2024

Related U.S. Application Data
(60) Provisional application No. 63/350,768, filed on Jun. 9, 2022.

(51) Int. Cl. A61B 34/10; A61B 90/00; G06V 10/82

(52) U.S. Cl. CPC A61B 34/10; A61B 90/36; G06V 10/82; A61B 2034/105; A61B 2034/107; A61B 2090/365

(57) ABSTRACT

A method for surgical planning includes obtaining, by a. computing system, a. first point cloud representing a first portion of a bone or a first cloud representing at least a portion of a bone, applying, by the computing system, a point cloud neural network to generate a second point cloud based on the first point cloud, the second point cloud comprising at least one of points representing at least a second portion of the bone or points representing an axis along the bone, and generating, by the computing system, surgical planning information based on the second point cloud.

This application claims priority to U.S. Provisional Patent Application 63/350,768, filed Jun. 9, 2022, the entire content of which is incorporated by reference.

### BACKGROUND

Orthopedic surgeries often involve implanting one or more orthopedic prostheses into a patient. For example, in a total shoulder replacement surgery, a surgeon may attach orthopedic prostheses to a scapula and a humerus of a patient. In an ankle replacement surgery, a surgeon may attach orthopedic prostheses to a tibia and a talus of a patient. When planning an orthopedic surgery, it may be important for the surgeon to determine correct size, shape, etc. of bone.

### SUMMARY

This disclosure describes example techniques for determining bone characteristics (e.g., size, shape, location, etc.) of a portion of a bone for which there may not be available in image content. For instance, a pre-operative scan of a portion of the bone may be available when a surgeon is planning a surgery. However, for pre-operative surgical planning, it may be beneficial to have image content representing other portions of the bone, or possibly the entire bone, but such image content may not be available. This disclosure describes example techniques in which a computing system obtaining a first point cloud representing a first portion of a bone (e.g., less than the entirety of the bone), and utilizing one or more point cloud neural networks (PCNNs) to generate a second point cloud based on the first point cloud. The second point cloud may include points representing at least a second portion of the bone for which image content is not available. In some examples, the second point cloud may include points representing the entire bone.

In one or more examples, the computing system may utilize the generated second point cloud representing the second portion of the bone (e.g., for which image content is not available) for surgical planning. For instance, the computing system may generate information indicative of an axis for aligning an implant based on the second point cloud.

In some examples, rather than or in addition to generating the second point cloud representing the portion of the bone for which image content is not available, the computing system may directly generate points representing an axis along the bone. That is, the computing system may generate a second cloud that includes points representing an axis along the bone. In this way, in some examples, it may be possible to bypass the reconstruction of the other portions of the bone.

Accordingly, in one or more examples, the computing system may obtain a first point cloud representing a portion of a bone, and apply a point cloud neural network to generate a second point cloud based on the first point cloud. In one example, the second point cloud includes points representing at least a second portion of the bone (e.g., a portion for which image content is not available). In one example, the second point cloud includes points representing an axis along the bone (e.g., an axis for aligning an implant).

In one example, this disclosure describes a method for surgical planning, the method comprising: obtaining, by a computing system, a first point cloud representing a first portion of a bone; applying, by the computing system, a point cloud neural network to generate a second point cloud based on the first point cloud, the second point cloud comprising points representing at least a second portion of the bone; and generating, by the computing system, surgical planning information based on the second point cloud.

In one example, this disclosure describes a method for surgical planning, the method comprising: obtaining, by a computing system, a first point cloud representing at least a portion of a bone; applying, by the computing system, a point cloud neural network to generate a second point cloud based on the first point cloud, the second point cloud comprising points representing an axis along the bone; and generating, by the computing system, surgical planning information based on the second point cloud.

In one example, the disclosure describes a system comprising: a storage system configured to store a first point cloud representing a first portion of a bone of a patient; and processing circuitry configured to: obtain the first point cloud representing the first portion of the bone; apply a point cloud neural network to generate a second point cloud based on the first point cloud, the second point cloud comprising points representing at least a second portion of the bone; and generate surgical planning information based on the second point cloud.

In one example, the disclosure describes a system comprising: a storage system configured to store a first point cloud representing at least a portion of a bone of a patient; and processing circuitry configured to: obtain the first point cloud representing at least the portion of the bone; apply a point cloud neural network to generate a second point cloud based on the first point cloud, the second point cloud comprising points representing an axis along the bone; and generate surgical planning information based on the second point cloud.

The details of various examples of the disclosure are set forth in the accompanying drawings and the description below. Various features, objects, and advantages will be apparent from the description, drawings, and claims.

### BRIEF DESCRIPTION OF DRAWINGS

FIG. 1 is a block diagram illustrating an example system that may be used to implement the techniques of this disclosure.

FIG. 2 is a block diagram illustrating example components of a planning system, in accordance with one or more techniques of this disclosure.

FIG. 3 is a conceptual diagram illustrating an example point cloud neural network (PCNN), in accordance with one or more techniques of this disclosure.

FIG. 4 is a flowchart illustrating an example architecture of a T-Net model in accordance with one or more techniques of this disclosure.

FIG. 5 is a conceptual diagram illustrating a tibia, different portions of the tibia, and axis for aligning an implant, in accordance with one or more techniques of this disclosure.

FIG. 6 is a flowchart illustrating an example process for surgical planning, in accordance with one or more techniques of this disclosure.

FIG. 7 is another flowchart illustrating an example process for surgical planning, in accordance with one or more techniques of this disclosure.

FIG. 8 is a conceptual diagram illustrating a tibia and examples of knee spines.

FIG. 9 is a conceptual diagram illustrating a tibia plafond landmark.

FIG. 10 is a conceptual diagram illustrating another perspective of the knee spines.

### DETAILED DESCRIPTION

For various types of orthopedic surgeries, a surgeon may utilize image content representing different anatomical objects (e.g., bones) for surgical planning. As one example, the presence of the knee in the pre-operative CT scan is an element for a correct planning of a total ankle replacement (TAR) surgery. For example, a tibia implant may be lined up on the tibia mechanical axis that is defined as the line passing through the tibia plafond landmark and the center of the proximal tibia (e.g., knee) spines. Without a knee model, there may be challenges to accurately plan the surgery, and there can possibly be an increase in the risks of potential complications leading to a premature later surgery.

However, in some cases, the image content of the bone useful for planning surgery may not be available. For example, image content (e.g., represented by a first point cloud) may be available for a first portion of the bone (e.g., distal end of the tibia), but may not be available for a second portion of the bone (e.g., proximal end of the tibia). This disclosure describes example techniques for determining the image content for the second portion bone (e.g., image content of bone that is unavailable). For instance, a computing system (e.g., including processing circuitry) may be configured to generate a second point cloud based on the first point cloud, where the second point cloud includes points representing at least a second portion of the bone. As an example, the processing circuitry may be configured to reconstruct the proximal tibia for cases for which the proximal tibia is missing in the CT scan, or the image quality of the proximal tibia is poor.

As one example, the processing circuitry may be configured to apply a point cloud neural network (PCNN) to generate a second point cloud based on the first point cloud, where the second point cloud includes points representing at least a second portion of the bone. The PCNN may be considered as a point completion model, and the processing circuitry may be configured to train the PCNN on cases for which the distal and proximal tibia parts are available to check the ability of the PCNN to recover the proximal tibia. For instance, the processing circuitry may generate training datasets based on bones of historic patients, and train the point cloud neural network using the training datasets. In some examples, another PCNN (e.g., another model) may be used to locate the center of the knee spines and improve the quality of the TAR planning.

As one example, the knee spines may be the lateral intercondylar and the medial intercondylar. The center of the knee spines may be the center between the lateral intercondylar and the medial intercondylar. This center may be deduced from the picking of the two spines on the proximal tibia, and may be useful for TAR planning as the center of the knee spines provides one point on the mechanical axis of the tibia.

For instance, the processing circuitry may generate surgical planning information based on the second point cloud. As an example, to generate the surgical planning information, the processing circuitry may generate information indicative of an axis for aligning an implant based on the second point cloud. The point cloud neural network used to generate the second point cloud may be considered as a first point cloud neural network. To generate information indicative of the axis, the processing circuitry may apply a second point cloud neural network to at least the second point cloud to generate the information indicative of the axis.

As described above, an example of the surgical information may be the axis for aligning the implant that the processing circuitry determines from a point cloud representing the image content that is not available. However, in some examples, instead of or in addition to generating the point cloud representing the image content that is not available, the processing circuitry may use a point cloud neural network to directly determine the axis for aligning the implant. For instance, instead of reconstructing the proximal knee, the processing circuitry may use a point completion model (e.g., point cloud neural network) with output points lined up along the tibia mechanical axis.

Therefore, in some examples, the processing circuitry may obtain a first point cloud representing a portion of a bone, and apply a point cloud neural network to generate a second point cloud based on the first point cloud. In this example, the second point cloud includes points representing an axis along the bone. The processing circuitry may generate surgical planning information based on the second point cloud. As an example, the second point cloud includes points representing a tibia mechanical axis that forms a line passing through a tibia plafond landmark and a center of proximal tibia spines (e.g., knee spines).

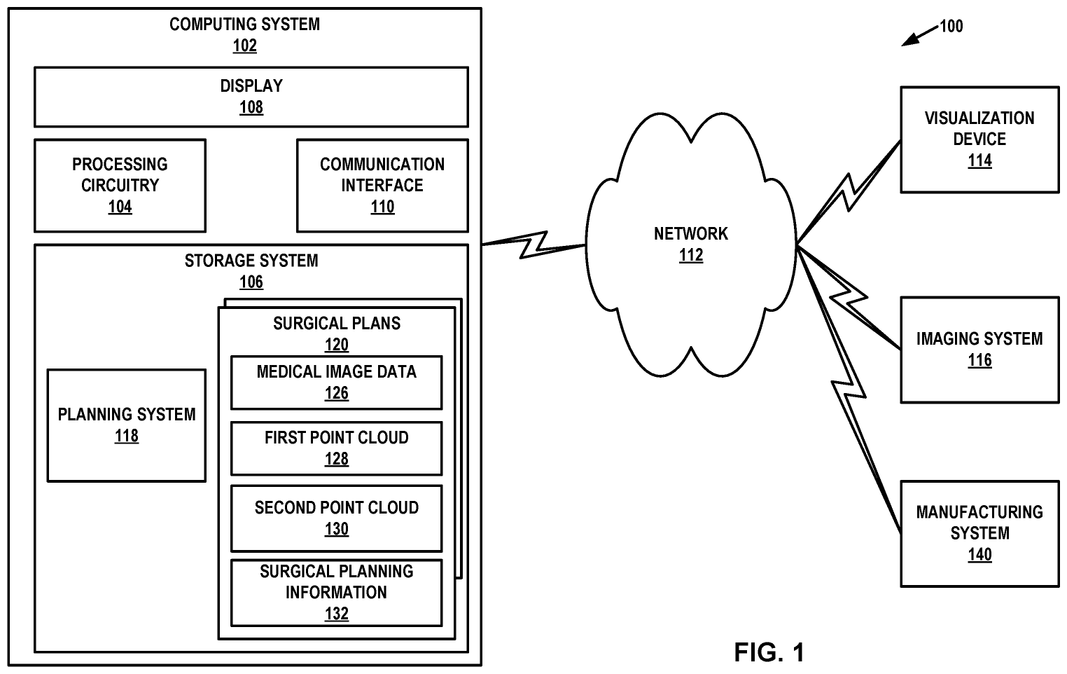

FIG. 1 is a block diagram illustrating an example system 100 that may be used to implement the techniques of this disclosure. In the example of FIG. 1, system 100 includes computing system 102, which is an example of one or more computing devices that are configured to perform one or more example techniques described in this disclosure. Computing system 102 may include various types of computing devices, such as server computers, personal computers, smartphones, laptop computers, and other types of computing devices. In some examples, computing system 102 includes multiple computing devices that communicate with each other. In other examples, computing system 102 includes only a single computing device. Computing system 102 includes processing circuitry 104, storage system 106, a display 108, and a communication interface 110. Display 108 is optional, such as in examples where computing system 102 is a server computer.

Examples of processing circuitry 104 include one or more microprocessors, digital signal processors (DSPs), application specific integrated circuits (ASICs), field programmable gate arrays (FPGAs), discrete logic, software, hardware, firmware or any combinations thereof. In general, processing circuitry 104 may be implemented as fixed-function circuits, programmable circuits, or a combination thereof. Fixed-function circuits refer to circuits that provide particular functionality and are preset on the operations that can be performed. Programmable circuits refer to circuits that can be programmed to perform various tasks and provide flexible functionality in the operations that can be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. In some examples, the one or more of the units may be distinct circuit blocks (fixed-function or programmable), and in some examples, the one or more units may be integrated circuits. In some examples, processing circuitry 104 is dispersed among a plurality of computing devices in computing system 102 and visualization device 114. In some examples, processing circuitry 104 is contained within a single computing device of computing system 102.

Processing circuitry 104 may include arithmetic logic units (ALUs), elementary function units (EFUs), digital circuits, analog circuits, and/or programmable cores, formed from programmable circuits. In examples where the operations of processing circuitry 104 are performed using software executed by the programmable circuits, storage system 106 may store the object code of the software that processing circuitry 104 receives and executes, or another memory within processing circuitry 104 (not shown) may store such instructions. Examples of the software include software designed for surgical planning.

Storage system 106 may be formed by any of a variety of memory devices, such as dynamic random access memory (DRAM), including synchronous DRAM (SDRAM), magnetoresistive RAM (MRAM), resistive RAM (RRAM), or other types of memory devices. Examples of display 108 include a liquid crystal display (LCD), a plasma display, an organic light emitting diode (OLED) display, or another type of display device. In some examples, storage system 106 may include multiple separate memory devices, such as multiple disk drives, memory modules, etc., that may be dispersed among multiple computing devices or contained within the same computing device.

Communication interface 110 allows computing system 102 to communicate with other devices via network 112. For example, computing system 102 may output medical images, images of segmentation masks, and other information for display. Communication interface 110 may include hardware circuitry that enables computing system 102 to communicate (e.g., wirelessly or using wires) with other computing systems and devices, such as a visualization device 114 and an imaging system 116. Network 112 may include various types of communication networks including one or more wide-area networks, such as the Internet, local area networks, and so on. In some examples, network 112 may include wired and/or wireless communication links.

Visualization device 114 may utilize various visualization techniques to display image content to a surgeon. In some examples, visualization device 114 is a computer monitor or display screen. In some examples, visualization device 114 may be a mixed reality (MR) visualization device, virtual reality (VR) visualization device, holographic projector, or other device for presenting extended reality (XR) visualizations. For instance, in some examples, visualization device 114 may be a Microsoft HOLOLENS™ headset, available from Microsoft Corporation, of Redmond, Washington, USA, or a similar device, such as, for example, a similar MR visualization device that includes waveguides. The HOLOLENS™ device can be used to present 3D virtual objects via holographic lenses, or waveguides, while permitting a user to view actual objects in a real-world scene, i.e., in a real-world environment, through the holographic lenses. In some examples, there may be multiple visualization devices for multiple users.

Visualization device 114 may utilize visualization tools that are available to utilize patient image data to generate three-dimensional models of bone contours, segmentation masks, or other data to facilitate preoperative planning. These tools may allow surgeons to design and/or select surgical guides and implant components that closely match the patient's anatomy. These tools can improve surgical outcomes by customizing a surgical plan for each patient. An example of such a visualization tool is the BLUEPRINT™ system available from Stryker Corp. The surgeon can use the BLUEPRINT™ system to select, design or modify appropriate implant components, determine how best to position and orient the implant components and how to shape the surface of the bone to receive the components, and design, select or modify guides or instruments to carry out the surgical plan. The information generated by the BLUEPRINT™ system may be compiled in a preoperative surgical plan for the patient that is stored in a database at an appropriate location, such as storage system 106, where the preoperative surgical plan can be accessed by the surgeon or other care provider, including before and during the actual surgery.

Imaging system 116 may comprise one or more devices configured to generate medical image data. For example, imaging system 116 may include a device for generating CT images. In some examples, imaging system 116 may include a device for generating MRI images. Furthermore, in some examples, imaging system 116 may include one or more computing devices configured to process data from imaging devices in order to generate medical image data. For example, the medical image data may include a 3D image of one or more bones of a patient. In this example, imaging system 116 may include one or more computing devices configured to generate the 3D image based on CT images or MRI images.

Computing system 102 may obtain a point cloud representing one or more bones of a patient. The point cloud may be generated based on the medical image data generated by imaging system 116. In some examples, imaging system 116 may include one or more computing devices configured to generate the point cloud. Imaging system 116 or computing system 102 may generate the point cloud by identifying the surfaces of the one or more bones in images and sampling points on the identified surfaces. Each point in the point cloud may correspond to a set of 3D coordinates of a point on a surface of a bone of the patient. In other examples, computing system 102 may include one or more computing devices configured to generate the medical image data based on data from devices in imaging system 116.

In one or more examples described in this disclosure, rather than having the entirety of a bone, imaging system 116 may have captured image content for a first portion of the bone (e.g., less than the entirety of the bone). Accordingly, computing system 102 may obtain a first point cloud representing a first portion of the bone. However, there may be instances where having image content for a second portion of the bone is desirable for surgical planning. In one or more examples, computing system 102 may be configured to generate a second point cloud based on the first point cloud, where the second point cloud includes points representing at least a second portion of the bone (e.g., the portion of the bone for which image content is unavailable). For instance, the first point cloud may exclude points for the second portion of the bone, and the example techniques may generate these points for the second portion of the bone. In some examples, the second point cloud may include the second portion of the bone, and additional portions of the bone, including the entirety of the bone. That is, the second point cloud may include points representing an entirety of the bone, including the second portion of the bone.

As described in more detail elsewhere in this disclosure, in some examples, the point cloud representing the second portion of the bone may be used for generating an axis for aligning an implant (e.g., a tibia mechanical axis that forms a line passing through a tibia plafond landmark and a center of proximal tibia spines). In some examples, rather than or in addition to, generating the second portion of the bone, computing system 102 may generate an axis for aligning an implant directly from the first point cloud (e.g., without needing the points representing the second portion of the bone).

Storage system 106 of computing system 102 may store instructions that, when executed by processing circuitry 104, cause computing system 102 to perform various activities. For instance, in the example of FIG. 1, storage system 106 may store instructions that, when executed by processing circuitry 104, cause computing system 102 to perform activities associated with a planning system 118. For ease of explanation, rather than discussing computing system 102 performing activities when processing circuitry 104 executes instructions, this disclosure may simply refer to planning system 118 or components thereof as performing the activities or may directly describe computing system 102 as performing the activities.

In the example of FIG. 1, storage system 106 stores surgical plans 120. Surgical plans 120 may correspond to individual patients. A surgical plan corresponding to a patient may include data associated with a planned or completed orthopedic surgery on the corresponding patient. A surgical plan corresponding to a patient may include medical image data 126 for the patient, first point cloud 128, second point cloud 130, and surgical planning information 132 for the patient. Medical image data 126 may include computed tomography (CT) images of bones of the patient or 3D images of bones of the patient based on CT images. In some examples, medical image data 126 may include magnetic resonance imaging (MRI) images of one or more bones of the patient or 3D images based on MRI images of the one or more bones of the patient. In some examples, medical image data 126 may include ultrasound images of one or more bones of the patient.

First point cloud 128 may represent a first portion of a bone. For instance, medical image data 126 may include image content for a bone, but in some cases, rather than having information for the entirety of the bone, medical image data 126 may include image content for a first portion of the bone (e.g., less than entirety of the bone). An example of the first portion of bone may be the distal tibia. Accordingly, first point cloud 128 may include points representing a first portion of the bone.

As another example, the example techniques may be useful for total knee arthroplasty (TKA). For TKA, some image content of the knee may be available, but image content of the hip and/or ankle may be missing or of poor image quality. For images of the knee, such joints of the knee, it may be possible to determine the ankle center and the hip center using example techniques described in this disclosure. The ankle center and/or hip center may be useful for the mechanical axis of the tibia and the femur.

As another example, the example techniques may be useful for total hip replacement (THR). For THR, the imagen content of the knee may be unavailable or of poor quality, but the image content of the hip and/or ankle is available. It may be possible to determine the hip from knee using example techniques described in this disclosure. The knee may be useful for determining the femur axis in THR.

In one example, second point cloud 130 may represent at least a second portion of the bone (e.g., at least some of the portion of the bone for which image content is unavailable). It may be possible for second point cloud 130 to include the entirety of the bone as well. However, in some examples, second point cloud 130 may include points representing an axis along the bone. In examples where second point cloud 130 represents an axis along the bone, it may be possible for first point cloud 128 to include points representing just a portion of the bone or the entirety of the bone. That is, in examples where second point cloud 130 represents an axis along the bone, first point cloud 128 may represent at least a portion of the bone (e.g., some of the bone or all of the bone).

Planning system 118 may be configured to assist a surgeon with planning an orthopedic surgery. Planning system 118 may assist the surgeon by providing the surgeon with data regarding at least one of image content of the portion of the bone for which image content is not available and/or an axis along the bone. In accordance with one or more techniques of this disclosure, planning system 118 may apply a point cloud neural network (PCNN) to generate an output point cloud based on an input point cloud. First point cloud 128 may be the input point cloud and second point cloud 130 may be the output point cloud. As described, first point cloud 128 may represent at least a portion of a bone.

Planning system 118 may determine second point cloud 130. In some examples, second point cloud 130 may include points representing at least a second portion of the bone, from which it may be possible to determine an axis for aligning an implant (e.g., based on another PCNN). In some examples, second point cloud 130 may include points representing an axis along the bone (e.g., without necessarily needing to determine a second portion of the bone). The axis along the bone may be a tibia mechanical axis that forms a line passing through a tibia plafond landmark and a center of proximal tibia spines.

In the example of FIG. 1, system 100 includes a manufacturing system 140. Manufacturing system 140 may manufacture a patient-specific tool alignment guide, tools, or implant, such as based on the second point cloud 130. As one example, such as examples where second point cloud 130 includes points representing an axis along the bone, manufacturing system 140 may utilize the second point cloud 130 to determine (e.g., select or manufacture) an implant, guide, or tools so that the implant can be properly positioned along the axis. As an other example, such as examples where second point cloud 130 includes points representing a portion of the bone, manufacturing system 140 may utilize second point cloud 130, and possibly first point cloud 128, to determine (e.g., select or manufacture) an implant, guide, or tools so that the implant is properly sized to fit on the bone, and the incision location is accurate.

For example, manufacturing system 140 may comprise an additive manufacturing device (e.g., a 3D printer) configured to generate an implant, guide, or tool. In other examples, manufacturing system 140 may include other types of devices, such as a reductive manufacturing device, a molding device, or other types of devices to generate the implant, guide, or tool.

In one or more examples, planning system 118 may generate surgical planning information 132 based on second point cloud 130. For instance, in examples where second point cloud 130 includes points representing a second portion of the bone (e.g., portion of the bone for which image content is unavailable), surgical planning information 132 may be information indicative of an axis for aligning an implant based on second point cloud 130. As one example, the PCNN used to generate second point cloud 130 may be considered as a first PCNN. Planning system 118 may apply a second PCNN trained to determine the axis to at least second point cloud 130 to generate the information indicative of the axis, which is an example of surgical planning information 132. Another example of surgical planning information 132 may be information for a Mixed Reality visualization of at least the second portion of the bone.

In examples where second point cloud 130 includes points representing an axis along the bone, surgical planning information 132 may be information for a Mixed Reality visualization of at least the axis along the bone. In general, surgical planning information 132 may information used for pre-operative and/or intra-operative surgical planning.

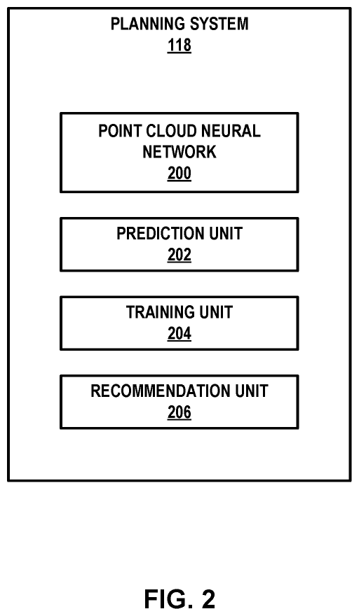

FIG. 2 is a block diagram illustrating example components of planning system 118, in accordance with one or more techniques of this disclosure. In the example of FIG. 2, the components of planning system 118 include a PCNN 200, a prediction unit 202, a training unit 204, and a recommendation unit 206. In other examples, planning system 118 may be implemented using more, fewer, or different components. For instance, training unit 204 may be omitted in instances where PCNN 200 has already been trained. In some examples, one or more of the components of planning system 118 are implemented as software modules. Moreover, the components of FIG. 2 are provided as examples and planning system 118 may be implemented in other ways.

Prediction unit 202 may apply PCNN 200 to generate an output point cloud based on an input point cloud. The input point cloud represents at least a first portion of a patient (e.g., first point cloud 128 of FIG. 1). In some examples, the output point cloud (e.g., second point cloud 130) includes points representing at least a second portion of the bone (e.g., portion of the bone for which image content is not available). In some examples, the output point cloud (e.g., second point cloud 130) includes points representing an axis along the bone. In examples where second point cloud 130 includes points representing an axis along the bone, the input point cloud (e.g., first point cloud 128) need not necessarily be of just a portion of the bone, and may include the entirety of the bone. That is, first point cloud 128 represents at least a portion of a bone, including the entirety of the bone.

Prediction unit 202 may obtain the input point cloud in one of a variety of ways. For example, prediction unit 202 may generate the input point cloud based on medical image data (e.g., medical image data 126 of FIG. 1). The medical image data for the patient may include a plurality of input images (e.g., CT images or MRI images, etc.). In this example, each of the input images may have a width dimension and a height dimension, and each of the input images may correspond to a different depth-dimension layer in a plurality of depth-dimension layers. In other words, the plurality of input images may be conceptualized as a stack of 2D images, where the positions of individual 2D images in the stack correspond to the depth dimension. As part of generating the point cloud, prediction unit 202 may perform an edge detection algorithm (e.g., Canny edge detection, Phase Stretch Transform (PST), etc.) on the 2D images (or a 3D image based on the 2D images). Prediction unit 202 may select points on the detected edges as points in the input point cloud. In other examples, prediction unit 202 may obtain the input point cloud from one or devices outside of computing system 102.

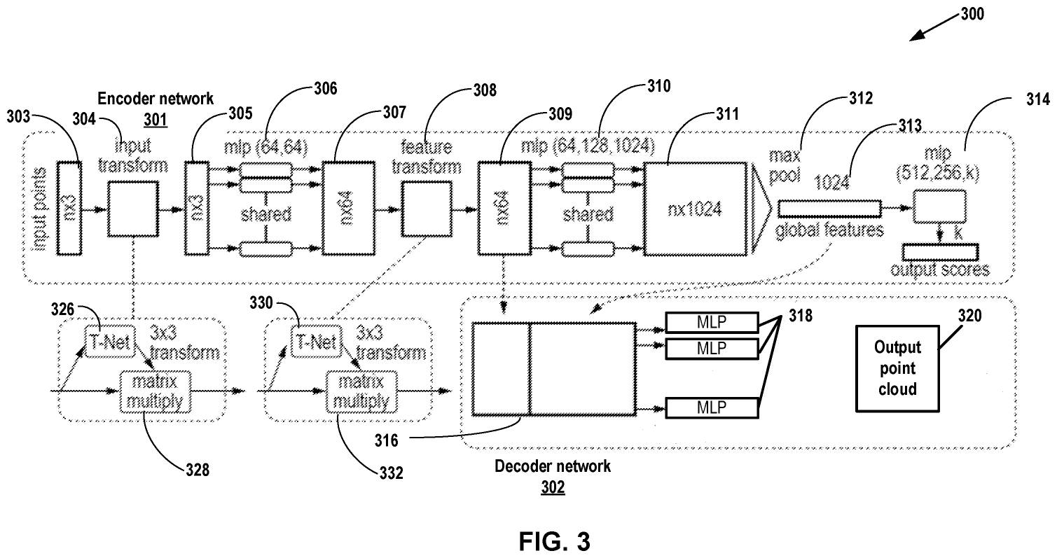

PCNN 200 is implemented using a point cloud learning model-based architecture. A point cloud learning model-based architecture (e.g., a point cloud learning model) is a neural network-based architecture that receives one or more point clouds as input and generates one or more point clouds as output. Example point cloud learning models include PointNet, PointTransformer, and so on. An example point cloud learning model-based architecture based on PointNet is described below with respect to FIG. 3.

Planning system 118 may include different sets of PCNNs for different surgery types. The set of PCNNs for a surgery type may include one or more PCNNs corresponding to different instances where the surgeon desires a representation of a portion of the bone for which image content is not available, and/or where the surgeon desires a representation of an axis along the bone. Furthermore, in examples where a first PCNN is used to generate points representing at least a second portion of the bone, planning system 118 may apply a second PCNN to at least the second point cloud to generate surgical planning information, such as information indicative of an axis for aligning an implant.

Training unit 204 may train PCNN 200. For instance, training unit 204 may generate a plurality of training datasets. Each of the training datasets may correspond to a different historic patient in a plurality of historic patients. The historic patients may include patients for whom image content of the bone is available, and patients for whom an axis on the bone for aligning an implant was previously determined. For instance, surgical plans 120 (FIG. 1) may include surgical plans for the historic patients. In some examples, the surgical plans may be limited to those developed by expert surgeons (e.g., to ensure high quality training data). In some examples, the historic patients may be selected for relevance.

The training dataset for a historic patient may include training input data and expected output data. The training input data may include a point cloud representing at least a first portion of the bone. In examples where PCNN 200 generates output point clouds indicating a second portion of the bone, the expected output data may be a point cloud that includes points indicating the second portion of the bone on the historic patient. In examples where PCNN 200 generates output point clouds representing an axis along the bone, the expected output data may comprise a point cloud that represents an axis along the bone that an expert surgeon had selected. In some examples, training unit 204 may generate the training input data based on medical image data stored in surgical plans of historic patients.

Training unit 204 may train PCNN 200 based on the training datasets. Because training unit 204 generates the training datasets based on how real surgeons actually planned and/or executed surgeries in historic patients, a surgeon who ultimately uses surgical planning information generated based on second point cloud 130 (e.g., output point cloud) may have confidence that the surgical planning information represents surgical planning information that expert surgeons would have generated.

In some examples, as part of training PCNN 200, training unit 204 may perform a forward pass on PCNN 200 using the input point cloud of a training dataset as input to PCNN 200. Training unit 204 may then perform a process that compares the resulting output point cloud generated by PCNN 200 to the corresponding expected output point cloud. In other words, training unit 204 may use a loss function to calculate a loss value based on the output point cloud generated by PCNN 200 and the corresponding expected output point cloud. In some examples, the loss function is targeted at minimizing a difference between the output point cloud generated by PCNN 200 and the corresponding expected output point cloud. Examples of the loss function may include a Chamfer Distance (CD) and the Earth Mover's Distance (EMD). The CD may be given by the average of a first average and a second average. The first average is an average of distances between each point in the output point cloud generated by PCNN 200 and its closest point in the expected output point cloud. The second average is an average of distances between each point in the expected output point cloud and its closest point in the output point cloud generated by PCNN 200. The CD may be defined as:

$${L}_{CD} ( {S}_{1} , {S}_{2} ) = \frac{1}{2} ( \frac{1}{\vert {S}_{1} \vert} {\sum }_{x ∈ {𝒮}_{1}} {min}_{y ∈ {𝒮}_{2}} ‖ x - y ‖ + \frac{1}{\vert {S}_{2} \vert} {\sum }_{y ∈ {𝒮}_{2}} {min}_{x ∈ {𝒮}_{1}} ‖ x - y ‖ )$$

In the equation above, S₁ is the output point cloud generated by PCNN 200, S₂ is the expected output point cloud, | . . . | is an element indicating number of elements, and ∥ . . . ∥ indicates absolute value.

Training unit 204 may then perform a backpropagation process based on the loss value to adjust parameters of PCNN 200 (e.g., weights of neurons of PCNN 200). In some examples, training unit 204 may determine an average loss value based on loss values calculated from output point clouds generated by performing multiple forward passes through PCNN 200 using different input point clouds of the training data. In such examples, training unit 204 may perform the backpropagation process using the average loss value to adjust the parameters of PCNN 200. Training unit 204 may repeat this process during multiple training epochs.

During use of PCNN 200 (e.g., after training of PCNN 200), prediction unit 202 of planning system 118 may apply PCNN 200 to generate an output point cloud for a patient based on an input point cloud representing a portion or at least a portion of a bone of the patient. In some examples, recommendation unit 206 may be configured to generate surgical planning information 132 based on the output point cloud (e.g., second point cloud 130). As one example, recommendation unit 206 may generate information indicative of an axis along the bone for aligning an implant based on the second point cloud. For example, recommendation unit 206 may utilize point cloud neural network 200 to apply another point cloud neural network to at least the second point cloud to generate the information indicative of the axis.

In some examples, recommendation unit 206 may generate information for a Mixed Reality visualization of at least the second portion of the bone (e.g., the portion of the bone for which image content is unavailable). In some examples, recommendation unit 206 may generate information for a Mixed Reality visualization of at least the axis along the bone (e.g., the axis for aligning an implant).

For instance, recommendation unit 206 may output for display one or more images (e.g., one or more 2D or 3D images) or models. For example, recommendation unit 206 may reconstruct a bone model from the points of first point cloud 128 and second point cloud 130 (e.g., by using points of the input point cloud as vertices of polygons, where the polygons form a hull of the bone model). In some examples, recommendation unit 206 may output for display a graphical representation of the axis along the bone for overlaying on the bone during surgery.

In this way, recommendation unit 206 may generate, based on second point cloud 130, information for a MR visualization. In examples where visualization device 114 (FIG. 1) is an MR visualization device, visualization device 114 may display the MR visualization. In some examples, visualization device 114 may display the MR visualization during a planning phase of a surgery. In such examples, recommendation unit 206 may generate the MR visualization as a 3D image in space. Recommendation unit 206 may generate the 3D image in the same as described above for generating the 3D image.

In some examples, the MR visualization is an intra-operative MR visualization. In other words, visualization device 114 may display the MR visualization during surgery. In some examples, visualization device 114 may perform a registration process that registers the MR visualization with the physical bones of the patient. Accordingly, in such examples, a surgeon wearing visualization device 114 may be able to see axis along the bone or the portion of the bone for which image content was not available on the bone.

FIG. 3 is a conceptual diagram illustrating an example point cloud learning model 300 in accordance with one or more techniques of this disclosure. Point cloud learning model 300 may receive an input point cloud. The input point cloud is a collection of points. The points in the collection of points are not necessarily arranged in any specific order. Thus, the input point cloud may have an unstructured representation.

In the example of FIG. 3, point cloud learning model 300 includes an encoder network 301 and a decoder network 302. Encoder network 301 receives an array 303 of n points. The points in array 303 may be the input point cloud of point cloud learning model 300. In the example of FIG. 3, each of the points in array 303 has a dimensionality of 3. For instance, in a Cartesian coordinate system, each of the points may have an x coordinate, a y coordinate, and a z coordinate.

Encoder network 301 may apply an input transform 304 to the points in array 303 to generate an array 305. Encoder network 301 may then use a first shared multi-layer perceptron (MLP) 306 to map each of the n points in array 305 from three dimensions to a larger number of dimensions a (e.g., a=64 in the example of FIG. 3), thereby generating an array 307 of n×a (e.g., n×64 values). For ease of explanation, the following description of FIG. 3 assumes that a is equal to 64 but in other examples other values of a may be used. Encoder network 301 may then apply a feature transform 308 to the values in array 307 to generate an array 309 of n×64 values. For each of the n points in array 309, encoder network 301 uses a second shared MLP 310 to map the n points from a dimension to b dimensions (e.g., b=1024 in the example of FIG. 3), thereby generating an array 311 of n×b (e.g., n×1024 values). For ease of explanation, the following description of FIG. 3 assumes that b is equal to 1024 but in other examples other values of b may be used. Encoder network 301 applies a max pooling layer 312 to generate a global feature vector 313. In the example of FIG. 3, each of points n in global feature vector 313 has 1024 dimensions.

Thus, as part of applying an PCNN 200, computing system 102 may apply an input transform (e.g., input transform 304) to a first array (e.g., array 303) that comprises the point cloud to generate a second array (e.g., array 305), wherein the input transform is implemented using a first T-Net model (e.g., T-Net Model 326), apply a first MLP (e.g., MLP 306) to the second array to generate a third array (e.g., array 307), apply a feature transform (e.g., feature transform 308) to the third array to generate a fourth array (e.g., array 309), wherein the input transform is implemented using a second T-Net model (e.g., T-Net model 330), apply a second MLP (e.g., MLP 310) to the fourth array to generate a fifth array (e.g., array 311), and apply a max pooling layer (e.g., max pooling layer 312) to the fifth array to generate the global feature vector (e.g., global feature vector 313).

A fully-connected network 314 may map global feature vector 313 to k output classification scores. The value k is an integer indicating a number of classes. E ach of the output classification scores corresponds to a different class. An output classification score corresponding to a class may indicate a level of confidence that the input point cloud as a whole corresponds to the class. Fully-connected network 314 includes a neural network having two or more layers of neurons in which each neuron in a layer is connected to each neuron in a subsequent layer. In the example of FIG. 3, fully-connected network 314 includes an input layer having 512 neurons, a middle layer having 256 neurons, and an output layer having k neurons. In some examples, fully-connected network 314 may be omitted from encoder network 301.

In some examples, input 316 to decoder network 302 may be formed by concatenating the n 64-dimensional points of array 309 with global feature vector 313. In other words, for each point of the n points in array 309, the corresponding 64 dimensions of the point are concatenated with the 1024 features in global feature vector 313. In some examples, array 309 is not concatenated with global feature vector 313. Decoder network 302 may sample N points in a unit square in 2-dimensions.

Thus, decoder network 302 may randomly determine N points having x-coordinates in a range of [0,1] and y-coordinates in the range of [0,1]. For each respective point of the N points, decoder network 302 may obtain a respective input vector by concatenating the respective point with global feature vector 313. Thus, in examples where array 309 is not concatenated with global feature vector 313, each of the input vectors may have 1026 features. For each respective input vector, decoder network 302 may apply each of K MLPs 318 (where K is an integer greater than or equal to 1) to the respective input vector. Each of MLPs 318 may correspond to a different patch (e.g., area) of the output point cloud. When decoder 302 applies the MLP to an input vector, the MLP may generate a 3-dimensional point in the patch (e.g., area) corresponding to the MLP. Thus, each of the MLPs 318 may reduce the number of features from 1026 to 3. The 3 features may correspond to the 3 coordinates of a point of the output point cloud. For instance, for each sampled point n in N, the MLPs 318 may reduce the features from 1026 to 512 to 256 to 128 to 64 to 3. Thus, decoder network 302 may generate a K×N×3 vector containing an output point cloud 320. In some examples, K=16 and N=512, resulting in second point cloud with 8192 3D points. In other examples, other values of K and N may be used. In some examples, as part of training the MLPs of decoder network 302, decoder network 302 may calculate a chamfer loss of an output point cloud relative to a ground-truth point cloud. Decoder network 302 may use the chamfer loss in a backpropagation process to adjust parameters of the MLPs. In this way, planning system 118 may apply the decoder (e.g., decoder network 302) to generate second point cloud data 130 representing at least a second portion of the bone or representing an axis along the bone based on the global feature vector.

In some examples, MLPs 318 may include a series of four fully-connected layers of neurons. For each of MLPs 318, decoder network 302 may pass an input vector of 1026 features to an input layer of the MLP. The fully-connected layers may reduce to number of features from 1026 to 512 to 256 to 3.

Input transform 304 and feature transform 308 in encoder network 301 may provide transformation invariance. In other words, point cloud learning model 300 may be able to generate output point clouds (e.g., second point cloud 130) in the same way, regardless of how the input point cloud (e.g., input bone model) is rotated, scaled, or translated. The fact that point cloud learning model 300 provides transform invariance may be advantageous because it may reduce the susceptibility of a generator ML model to errors based on positioning/scaling in morbid bone models. As shown in the example of FIG. 3, input transform 304 may be implemented using a T-Net Model 326 and a matrix multiplication operation 328. T-Net Model 326 generates a 3×3 transform matrix based on array 303. Matrix multiplication operation 328 multiplies array 303 by the 3×3 transform matrix. Similarly, feature transform 308 may be implemented using a T-Net model 330 and a matrix multiplication operation 332. T-Net model 330 may generate a 64×64 transform matrix based on array 307. Matrix multiplication operation 328 multiplies array 307 by the 64×64 transform matrix.

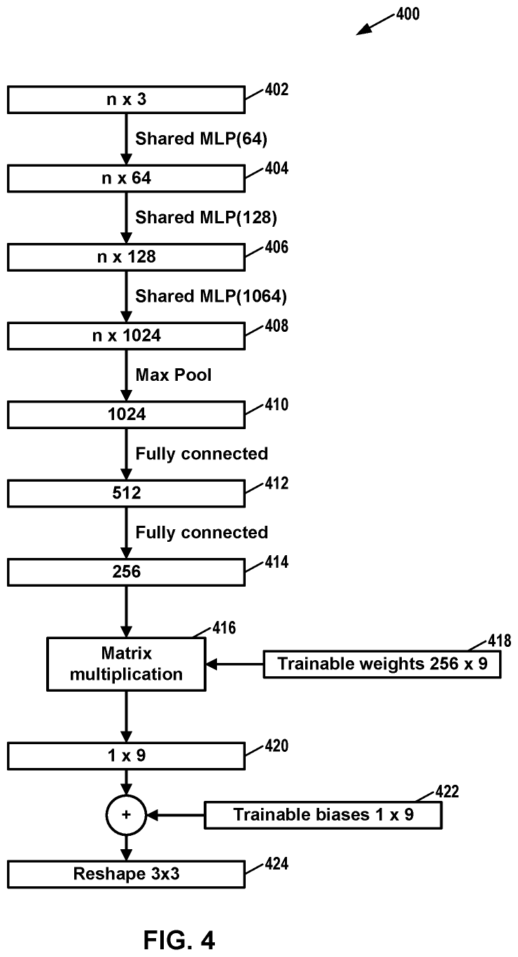

FIG. 4 is a block diagram illustrating an example architecture of a T-Net model 400 in accordance with one or more techniques of this disclosure. T-Net model 400 may implement T-Net Model 326 used in the input transform 304. In the example of FIG. 4, T-Net model 400 receives an array 402 as input. Array 402 includes n points. Each of the points has a dimensionality of 3. A first shared MLP maps each of the n points in array 402 from 3 dimensions to 64 dimensions, thereby generating an array 404. A second shared MLP maps each of the n points in array 404 from 64 dimensions to 128 dimensions, thereby generating an array 406. A third shared MLP maps each of the n points in array 406 from 128 dimensions to 1024 dimensions, thereby generating an array 408. T-Net model 400 then applies a max pooling operation to array 408, resulting in an array 810 of 1024 values. A first fully-connected neural network maps array 410 to an array 812 of 512 values. A second fully-connected neural network maps array 412 to an array 414 of 256 values. T-Net model 400 applies a matrix multiplication operation 416 to a matrix of trainable weights 418. The matrix of trainable weights 418 has dimensions of 256×9. Thus, multiplying array 414 by the matrix of trainable weights 418 results in an array 820 of size 1×9. T-Net model 400 may then add trainable biases 422 to the values in array 420. A reshaping operation 424 may remap the values resulting from adding trainable biases 422 into a 3×3 transform matrix. In other examples, the sizes of the matrixes and arrays may be different.

T-Net model 330 (FIG. 3) may be implemented in a similar way as T-Net model 400 in order to perform feature transform 308. However, in this example, the matrix of trainable weights 418 is 256×4096 and the trainable biases 422 has size 1×4096 bias values instead of 9. Thus, the T-Net model for performing feature transform 308 may generate a transform matrix of size 64×64. In other examples, the sizes of the matrixes and arrays may be different.

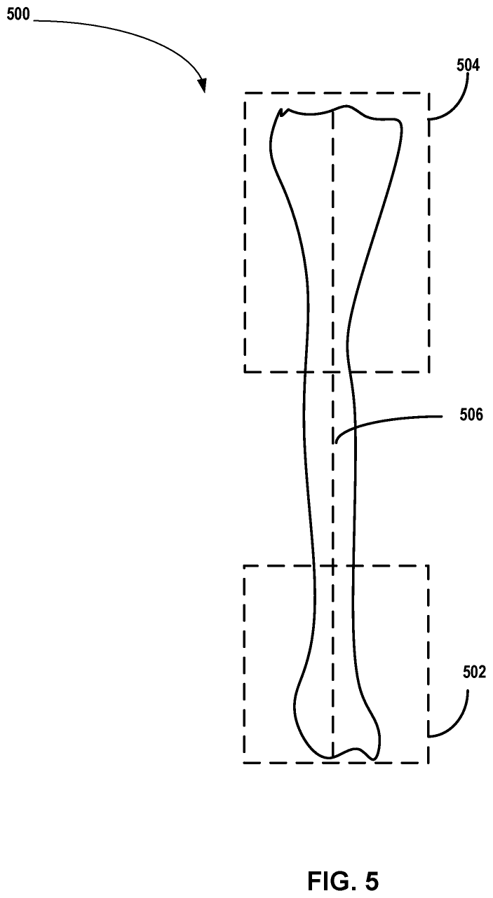

FIG. 5 is a conceptual diagram illustrating a tibia, different portions of the tibia, and axis for aligning an implant, in accordance with one or more techniques of this disclosure. For instance, FIG. 5 illustrates bone 500, which is a tibia. In FIG. 5, first portion 502 represents the distal end of the tibia, and in some examples, image content of first portion 502 may be available, but image content of other portions may not be available. For instance, in FIG. 5, second portion 504 represents the proximal end of the tibia (e.g., knee). In general, the more image content of first portion 502 that is available may result in better determination of the missing portion (or portion having poor image quality), such as second portion 504.

In one example, processing circuitry 104 of computing system 102 may obtain a first point cloud 128 representing first portion 502 of bone 500. First point cloud 128 may exclude points for second portion 504. As one example, first point cloud 128 includes points representing a distal end of the tibia.

Processing circuitry 104 may apply a point cloud neural network (e.g., one example of PCNN 200) to generate a second point cloud 130 based on the first point cloud 128. In this example, the second point cloud 130 includes points representing at least a second portion 504 of the bone 500. As one example, second point cloud 130 includes points representing a proximal end of the tibia. However, the example techniques are not so limited. In some examples, the second point cloud 130 may include points representing an entirety of bone 500, including the second portion 504 of bone 500.

Processing circuitry 104 may generate surgical planning information based on second point cloud 130. As one example, processing circuitry 104 may generate information indicative of an axis 506 along the bone 500 for aligning an implant based on the second point cloud 130. For instance, the point cloud neural network that processing circuitry 104 utilized to generate second point cloud 130 may be a first point cloud neural network. To generate information indicative of the axis 506, processing circuitry 104 may apply a second point cloud neural network to at least the second point cloud to generate the information indicative of the axis 506. For example, generating information indicative of the axis includes generating information indicative of a tibia mechanical axis that forms a line passing through a tibia plafond landmark and a center of proximal tibia spines (e.g., knee spines). As another example, generating the surgical planning information includes generating information for a Mixed Reality visualization of at least the second portion 504 of the bone 500.

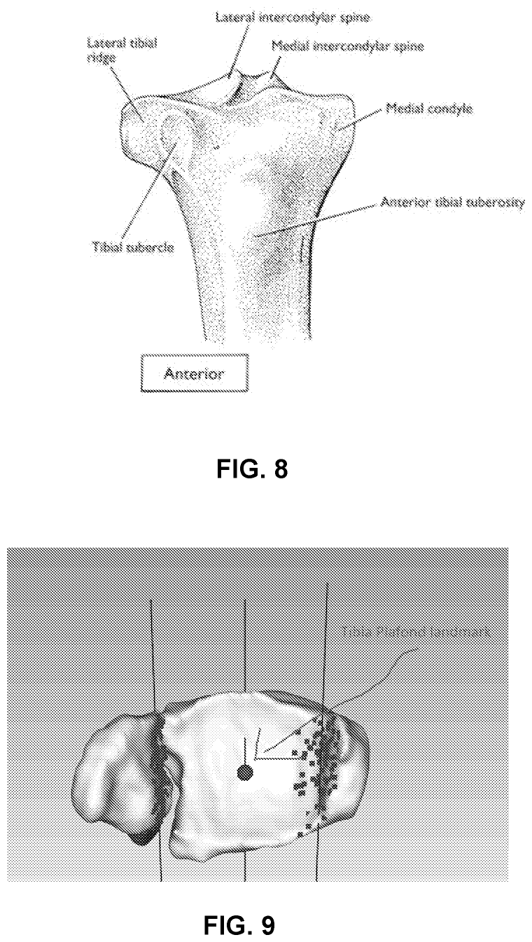

FIG. 8 is a conceptual diagram illustrating a tibia and examples of knee spines. FIG. 9 is a conceptual diagram illustrating a tibia plafond landmark. In FIG. 8, the lateral intercondylar spine and the medial intercondylar spine are examples of proximal tibia spines (e.g., knee spines). The center of knee spines may be the center between the intercondylar spine and the medial intercondylar spine. In one or more examples, axis 506 may be centered between the lateral intercondylar spine and the medial intercondylar spine and through the tibia plafond landmark shown in FIG. 9. The implant may be aligned with axis 506.

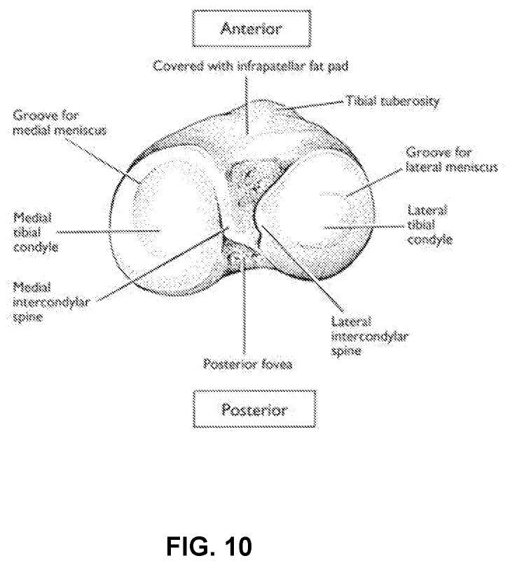

FIG. 10 is a conceptual diagram illustrating another perspective of the knee spines. For instance, in FIG. 10, the lateral intercondylar spine and the medial intercondylar spine are shown from the top perspective. The tibia plafond landmark may be between the lateral intercondylar spine and the medial intercondylar spine in FIG. 10.

Referring back to FIG. 5, in the above example, second point cloud 130 includes points representing at least a second portion 504 of bone 500. However, in some examples, rather than second point cloud 130 including points representing at least a second portion 504 of bone 500, processing circuitry 104 may apply a point cloud neural network (e.g., another example of PCNN 200) to generate a second point cloud 130 based on the first point cloud 128, where the second point cloud 130 includes points representing an axis 506 along the bone 500. That is, generation of points representing at least second portion 504 of bone 500 may be optional, and it may be possible for processing circuitry 104 to generate axis 506 without necessarily first generating points for second portion 504. In such examples, processing circuitry 104 may obtain a first point cloud 128 representing at least a portion of bone 500. For instance, in such examples, first point cloud 128 may include points representing only first portion 502, but may include more portions than only first portion 502, including the entire bone 500.

There may be various ways in which processing circuitry 104 may apply a point cloud neural network. As one example, to apply the point cloud neural network, processing circuitry 104 may apply an input transform to a first array that comprises the first point cloud to generate a second array, wherein the input transform is implemented using a first T-Net model, apply a first multi-layer perceptron (MLP) to the second array to generate a third array, apply a feature transform to the third array to generate a fourth array, wherein the input transform is implemented using a second T-Net model, apply a second MLP to the fourth array to generate a fifth array, apply a max pooling layer to the fifth array to generate a global feature vector, sample N points in a unit square in 2-dimensions, concatenate the sampled points with the global feature vector to obtain a combined vector, and apply one or more third MLPs to generate points in the second point cloud 130

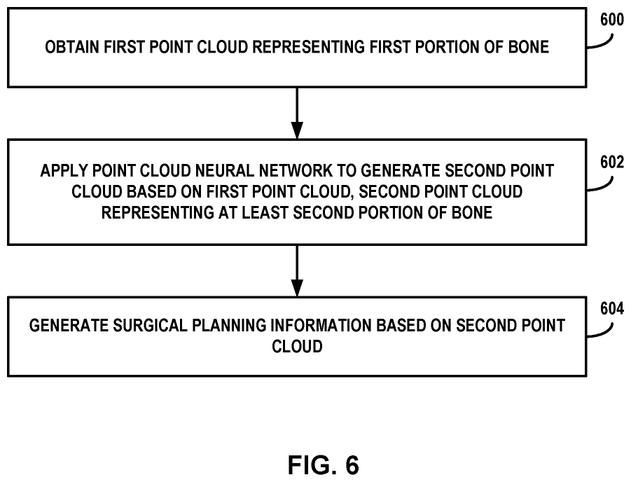

FIG. 6 is a flowchart illustrating an example process for surgical planning, in accordance with one or more techniques of this disclosure. Computing system 102 (e.g., via processing circuitry 104) may obtain a first point cloud 128 representing a first portion of a bone (600). One example of first portion is first portion 502 of bone 500 in FIG. 5. In some examples, obtaining the first point cloud includes obtaining the first point cloud that excludes points for a second portion of the bone (e.g., excludes points for second portion 504 of bone 500). First point cloud 128 may include points representing a distal end of a tibia.

Computing system 102 may apply a point cloud neural network to generate a second point cloud 130 based on the first point cloud 128, the second point cloud 130 including points representing at least a second portion of the bone (602). Second point cloud 130 may include points representing a proximate end of the tibia. One example of the second portion is second portion 504 of bone 500 in FIG. 5. In some examples, computing system 102 may apply the point cloud neural network to generate the second point cloud 130 based on the first point cloud 128, where the second point cloud 130 includes points representing an entirety of the bone, including the second portion of the bone.

Computing system 102 may generate surgical planning information based on the second point cloud 130 (604). For example, to generate the surgical planning information, computing system 102 may generate information indicative of an axis along the bone for aligning an implant based on the second point cloud 130. As one example, the point cloud neural network used to generate second point cloud 130 may be a first point cloud neural network. In some examples, to generate information indicative of the axis, computing system 102 may apply a second point cloud neural network (e.g. using techniques of FIG. 3 as described above) to at least the second point cloud 130 to generate the information indicative of the axis. As one example illustrated in FIG. 5, to generate information indicative of the axis 506, computing system 102 may generate information indicative of a tibia mechanical axis that forms a line passing through a tibia plafond landmark and a center of proximal tibia spines, as shown in FIG. 5.

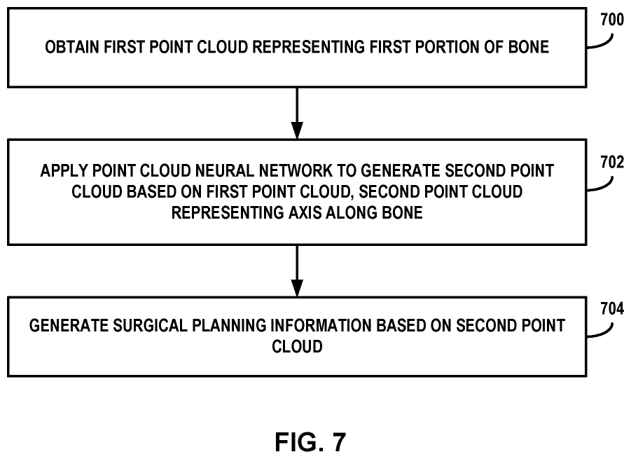

FIG. 7 is another flowchart illustrating an example process for surgical planning, in accordance with one or more techniques of this disclosure. Computing system 102 (e.g., via processing circuitry 104) may obtain a first point cloud 128 representing at least a portion of a bone (700). In one or more examples, obtaining the first point cloud 128 includes obtaining the first point cloud 128 that represents less than an entirety of the bone. In some examples, first point cloud 128 may include points representing a distal end of a tibia. However, the example techniques are not so limited. In some examples, for the example of FIG. 7, first point cloud 128 may include the entirety of the bone.

Computing system 102 may apply a point cloud neural network to generate a second point cloud 130 based on the first point cloud 128, where the second point cloud 130 includes points representing an axis along the bone (702). For example, to apply the point cloud neural network, computing system 102 may apply the point cloud neural network to generate the second point cloud 130 based on the first point cloud 128, where the second point cloud 130 includes points representing a tibia mechanical axis that forms a line passing through a tibia plafond landmark and a center of proximal tibia spines (e.g., as shown in FIG. 5).

Computing system 102 may generate surgical planning information based on the second point cloud (704). As one example, the surgical planning information may be information for a Mixed Reality visualization of at least the axis along the bone.

While the techniques been disclosed with respect to a limited number of examples, those skilled in the art, having the benefit of this disclosure, will appreciate numerous modifications and variations there from. For instance, it is contemplated that any reasonable combination of the described examples may be performed. It is intended that the appended claims cover such modifications and variations as fall within the true spirit and scope of the invention.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

In one or more examples, the functions described may be implemented in hardware, software, firmware, or any combination thereof. If implemented in software, the functions may be stored on or transmitted over as one or more instructions or code on a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Operations described in this disclosure may be performed by one or more processors, which may be implemented as fixed-function processing circuits, programmable circuits, or combinations thereof, such as one or more digital signal processors (DSPs), general purpose microprocessors, application specific integrated circuits (ASICs), field programmable gate arrays (FPGAs), or other equivalent integrated or discrete logic circuitry. Fixed-function circuits refer to circuits that provide particular functionality and are preset on the operations that can be performed. Programmable circuits refer to circuits that can programmed to perform various tasks and provide flexible functionality in the operations that can be performed. For instance, programmable circuits may execute instructions specified by software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. Accordingly, the terms “processor” and “processing circuitry,” as used herein may refer to any of the foregoing structures or any other structure suitable for implementation of the techniques described herein.

1.-10. (canceled)

11. A method for surgical planning, the method comprising:
obtaining, by a computing system, a first point cloud representing at least a portion of a bone;
applying, by the computing system, a point cloud neural network to generate a second point cloud based on the first point cloud, the second point cloud comprising points representing an axis along the bone; and
generating, by the computing system, surgical planning information based on the second point cloud.

12. The method of claim 11, wherein applying the point cloud neural network comprises:
applying the point cloud neural network to generate the second point cloud based on the first point cloud, the second point cloud comprising points representing a tibia mechanical axis that forms a line passing through a tibia plafond landmark and a center of proximal tibia spines.

13. The method of claim 11, wherein the first point cloud represents less than an entirety of the bone.

14. The method of claim 11, wherein the bone comprises a tibia, wherein the first point cloud comprises points representing a distal end of the tibia.

15. The method of claim 11, wherein generating the surgical planning information comprises generating information for a Mixed Reality visualization of at least the axis along the bone.

16. The method of claim 11, wherein applying the point cloud neural network comprises:
applying an input transform to a first array that comprises the first point cloud to generate a second array, wherein the input transform is implemented using a first T-Net model;
applying a first multi-layer perceptron (MLP) to the second array to generate a third array;
applying a feature transform to the third array to generate a fourth array, wherein the input transform is implemented using a second T-Net model;
applying a second MLP to the fourth array to generate a fifth array;
applying a max pooling layer to the fifth array to generate a global feature vector;
sampling N points in a unit square in 2-dimensions;
concatenating the sampled points with the global feature vector to obtain a combined vector; and
applying one or more third MLPs to generate points in the second point cloud.

17. The method of claim 11, further comprising training the point cloud neural network, wherein training the point cloud neural network comprises:
generating training datasets based on bones of historic patients; and
training the point cloud neural network using the training datasets.

18.-27. (canceled)

28. A system comprising:
a storage system configured to store a first point cloud representing at least a portion of a bone of a patient; and
processing circuitry configured to:
apply a point cloud neural network to generate a second point cloud based on the first point cloud, the second point cloud comprising points representing an axis along the bone; and
generate surgical planning information based on the second point cloud.

29. The system of claim 28, wherein to apply the point cloud neural network, the processing circuitry is configured to:
apply the point cloud neural network to generate the second point cloud based on the first point cloud, the second point cloud comprising points representing a tibia mechanical axis that forms a line passing through a tibia plafond landmark and a center of proximal tibia spines.

30. The system of claim 28, wherein the first point cloud represents less than an entirety of the bone.

31. The system of claim 28, wherein the bone comprises a tibia, wherein the first point cloud comprises points representing a distal end of the tibia.

32. The system of claim 28, wherein to generate the surgical planning information, the processing circuitry is configured to generate information for a Mixed Reality visualization of at least the axis along the bone.

33. The system of claim 28, wherein to apply the point cloud neural network, the processing circuitry is configured to:
apply an input transform to a first array that comprises the first point cloud to generate a second array, wherein the input transform is implemented using a first T-Net model;
apply a first multi-layer perceptron (MLP) to the second array to generate a third array;
apply a feature transform to the third array to generate a fourth array, wherein the input transform is implemented using a second T-Net model;
apply a second MLP to the fourth array to generate a fifth array;
apply a max pooling layer to the fifth array to generate a global feature vector;
sample N points in a unit square in 2-dimensions;
concatenate the sampled points with the global feature vector to obtain a combined vector; and
apply one or more third MLPs to generate points in the second point cloud.

34. The system of claim 28, wherein the processing circuitry is configured to train the point cloud neural network, wherein to train the point cloud neural network, the processing circuitry is configured to:
generate training datasets based on bones of historic patients; and
train the point cloud neural network using the training datasets.

35. (canceled)

36. A non-transitory computer-readable storage medium storing instructions thereon that when executed cause one or more processors to;
obtain a first point cloud representing at least a portion of a bone;
apply a point cloud neural network to generate a second point cloud based on the first point cloud, the second point cloud comprising points representing an axis along the bone; and
generate surgical planning information based on the second point cloud.

37. The non-transitory computer-readable storage medium of claim 36, wherein the instructions that cause the one or more processors to apply the point cloud neural network comprise instructions that cause the one or more processors:
apply the point cloud neural network to generate the second point cloud based on the first point cloud, the second point cloud comprising points representing a tibia mechanical axis that forms a line passing through a tibia plafond landmark and a center of proximal tibia spines.

38. The non-transitory computer-readable storage medium of claim 36, wherein the first point cloud represents less than an entirety of the bone.

39. The non-transitory computer-readable storage medium of claim 36, wherein the bone comprises a tibia, wherein the first point cloud comprises points representing a distal end of the tibia.

40. The non-transitory computer-readable storage medium of claim 36, wherein the instructions that cause the one or more processors to generate the surgical planning information comprise instructions that cause the one or more processors to generate information for a Mixed Reality visualization of at least the axis along the bone.

41. The non-transitory computer-readable storage medium of claim 36, wherein the instructions that cause the one or more processors to apply the point cloud neural network comprise instructions that cause the one or more processors:
apply an input transform to a first array that comprises the first point cloud to generate a second array, wherein the input transform is implemented using a first T-Net model;
apply a first multi-layer perceptron (MLP) to the second array to generate a third array;
apply a feature transform to the third array to generate a fourth array, wherein the input transform is implemented using a second T-Net model;
apply a second MLP to the fourth array to generate a fifth array;
apply a max pooling layer to the fifth array to generate a global feature vector;
sample N points in a unit square in 2-dimensions;
concatenate the sampled points with the global feature vector to obtain a combined vector; and
apply one or more third MLPs to generate points in the second point cloud.
